(19) United States
(12) Patent Application Publication
(10) Pub. No.: US 2010/0130878 A1
(43) Pub. Date: May 27, 2010

(54) SYSTEMS, APPARATUS AND PROCESSES FOR AUTOMATED BLOOD FLOW ASSESSMENT OF VASCULATURE

(75) Inventors: Andras Lasso, Budapest (HU); Ferenc Nasztanovics, Budapest (HU)

Correspondence Address: DEAN D. SMALL;THE SMALL PATENT LAW GROUP LLP, 225 S. MERAMEC, STE. 725T, ST. LOUIS, MO, 63105, US

(73) Assignee: GENERAL ELECTRIC COMPANY, Schenectady, NY (US)

(21) Appl. No.: 12/276,483

(22) Filed: Nov. 24, 2008

(51) Int. Cl. A61B 5/026; G06Q 50/00; G06F 17/10; G06G 7/57; G06G 7/60; A61B 5/02

(52) U.S. Cl. 600/500; 705/3; 703/2; 703/9; 703/11; 600/504

(57) ABSTRACT

A system, apparatus and process for characterizing aspects of vascular scenarios is described, and includes an input module and a database. The system also includes access to a FSI solver. The FSI solver accepts information from the input module and the database, and uses the accepted information to model a vascular site of interest and provide results from modeling the vascular site of interest. The system also includes interfaces for transmitting information from the input module and the database to the FSI solver and for receiving the results from the FSI solver, and an ensemble of analysis modules which is coupled to the interface for receiving results. The ensemble of analysis modules compares various treatment options, allows before-and-after comparisons of aspects of the vascular site of interest and provides quantitative assessments of parameters of interest describing the vascular site of interest.

### FIELD OF THE DISCLOSURE

This disclosure relates generally to anatomical data processing technology, and in particular to systems, apparatus and processes for accurately, rapidly, efficiently and robustly characterizing blood flow data and risk of vascular accident by using a situationally-variable, tailored blend of measured data and stored information, via a flexible, automated content enhancement tool.

### BACKGROUND

Stroke and cerebrovascular diseases are a major cause of premature death, and also represent a leading cause of major disability in the United States, Canada and Japan, among others. Hemorrhagic strokes account for a substantial minority of all stroke cases, and involve bleeding into the brain. In turn, of those strokes which are hemorrhagic, as opposed to occlusive (i.e., caused by an obstruction, such as a blood clot, blocking blood flow to a portion of the brain), one-third to two-thirds may result in death. A substantial portion of non-fatal hemorrhagic strokes, believed to be in a range of from about ten percent to about twenty percent of all hemorrhagic strokes, result in severe brain damage. In turn, such cerebral vascular accidents give rise to need for intense therapy, and frequently necessitate long-term care, due to often-irreversible brain damage. Many of these hemorrhagic strokes are due to rupture of intracranial aneurysms.

Epidemiological evidence suggests that a large majority of intracranial aneurysms do not rupture. When considering which aneurysms to treat, and in the selection of suitable treatment methods, a physician must attempt to estimate the likelihood of rupture, and, when deemed warranted and appropriate, the relative risks associated with the various candidate mechanisms and approaches for attempting intervention or repair. Current recommendations are primarily based on patient factors (such as aneurismal subarachnoid hemorrhage, age, and other relevant medical conditions), aneurysm characteristics (including at least size, location and morphology) and management factors (e.g., experience of the surgical team, etc.). Although the aneurysm characteristics employed to date in making such decisions are relatively easily measured, they offer a very limited description of the relevant aneurysm characteristics, and they utilize a small fraction of information that frequently is already available from the acquired diagnostic data and images.

As a result, there are numerous difficult problems that cannot be effectively addressed though use of currently available tools. Examples of such limitations and drawbacks to the prior art approaches include high-risk cases, such as giant aneurysms, where standard recommendations have limited applicability. Consequently, in such instances, an individualized determination of relative risks is desirable.

While many new technological advances offer previously unknown treatment options, including advances in coil technology, liquid polymer techniques, balloons, stents, surgical equipment, techniques, and the like, this increased range of available treatment tools also increases the complexity involved in determining suitable, presently-realizable options for recommendation, and further in attempting to rank-order those to determine an preferred option or range of options as candidates for employment in a particular patient and presenting condition. Ideally, selection of preferred treatment tools and methods for each patient, and estimation of probabilities associated with pre-treatment, intra-treatment and post-treatment threats to life or health, should be based on assessment of the applicability of the available tools for the particular patient, the presenting aneurysm profile and other relevant factors.

Also, increasing the degree of post-treatment aneurysm occlusion strongly correlates with reduced risk of re-rupture. In turn, this justifies attempts to completely occlude those aneurysms which are deemed candidates for invasive treatment. However, case reports have shown that even aneurysms that appear to be completely occluded after surgery, or endovascular coil embolization, may later rupture.

Although evidence suggests that one-year outcomes in patients with a ruptured aneurysm may be better after endovascular coiling than after surgical clipping, the long-term efficacy of coiling versus clipping remains uncertain. Recent prospective cohort studies have found reassuringly low rates of rehemorrhage with both surgical and endovascular techniques. Despite such low rates, the consequences of rehemorrhage can be devastating—mortality is greater than 50%. Focus has, therefore, turned towards better understanding the factors that may predispose to rehemorrhage and identifying the best methods for surveillance.

Improving pre-operative planning and/or intra-operative assessment of expected final occlusion thus may significantly reduce subsequent risk of rupture or re-rupture.

Similar challenges arise in related areas of diagnostic and medical intervention or treatment of other vascular diseases, such as abdominal aortic aneurysms (e.g., difficultly in estimating risk of rupture), carotid artery stenosis (for example, in realistically estimating risk of plaque rupture, erosion and thromboemboli formation) and heart valve diseases.

For the reasons stated above, and for other reasons discussed below, which will become apparent to those skilled in the art upon reading and understanding the present disclosure, there are needs in the art to provide new and more highly automated simulation and analysis tools for estimating the properties and propensities of a variety of vascular abnormalities with greater accuracy than has been possible heretofore, and for more generally-applicable protocols for application and usage of an increasing range of treatment aids and options, in order to streamline and improve usage of available information in forming risk assessments, together with an appropriate, comprehensive and readily updatable menu of treatment options for further consideration and ultimately for implementation of a chosen option or options, and for continued risk assessment after initiation of invasive or non-invasive treatment.

### BRIEF DESCRIPTION

The above-mentioned shortcomings, disadvantages and problems are addressed herein, which will be understood by reading and studying the following disclosure.

In one aspect, a system for characterizing aspects of vascular scenarios includes an input module and a database for storing characteristics of various types and conditions of vascular segments, a vascular site of interest and associated environments, properties of tools associated with treatment of vascular abnormalities, and patient-related information. The system also includes access to a FSI solver. The FSI solver accepts information from the input module and the database, and uses the accepted information to model the vascular site of interest and to provide results from modeling the vascular site of interest. The system also includes interfaces for transmitting information from the input module and the database to the FSI solver and for receiving the results from the FSI solver, and an ensemble of analysis modules which is coupled to the interface for receiving results. The ensemble of analysis modules is for comparing various treatment options, allowing before-and-after comparisons of aspects of the vascular site of interest and providing quantitative assessments of parameters of interest describing the vascular site of interest.

In another aspect, a process for characterizing aspects of vascular scenarios is described. The process includes acts of accepting patient indicia via an input module and accessing relevant data records from a database using the indicia. The process includes an act of augmenting those data records, where needed, with stored data from a bank of representative data also stored in the database, to provide information including a description of the vascular scenario and defining a region of interest. The process then includes an act of sending the information to a FSI solver, and an act of receiving, responsive to sending, raw simulation results from the FSI solver. The process further includes an act of modifying the raw simulation results using selected items from a collection of analysis modules. The selected items from the collection are for comparing various treatment options, allowing before-and-after comparisons of aspects of the region of interest and providing quantitative assessments of parameters of interest describing the region of interest from the results.

In a further aspect, the present disclosure teaches a computation engine and a memory coupled to a data collection module, and computer-readable code embodied on a computer-readable medium and configured so that when the computer-readable code is executed by one or more processors associated with the computation engine, the computer-readable code causes the one or more processors to perform acts including accepting input indicia via an input module. The input indicia identifies a particular patient and enables access to stored records relating to prior measurements and simulations, if any, relative to that patient. The computer-readable code is further configured, when executed by one or more processors, to cause the one or more processors to perform acts including determining estimates for quantities not represented in a present measurement by extracting suitable data from a database which stores characteristics of various types and conditions of vascular segments associated with a defined vascular region of interest and associated environments, and determining appropriate properties of tools associated with treatment of vascular abnormalities, in conformance with patient-related indicia, or information identifying such. The computer-readable code is additionally configured, when executed by one or more processors, to cause the one or more processors to perform acts including accessing a FSI solver. The FSI solver accepts an information including at least some of the characteristics, conditions, a description of the vascular region of interest and associated environments, the properties of tools associated with treatment of vascular abnormalities, and the patient-related indicia, or information identifying such from the input module and the database, and uses the accepted information to model the region of interest and provide results from modeling the region of interest. The computer-readable code also is configured, when executed by one or more processors, to cause the one or more processors to perform acts including exchanging information between the input module, the database and the FSI solver, including providing results from the FSI solver to a collection of analysis modules, and using the collection of analysis modules, and the results from the FSI solver to: compare benefits and potential drawbacks of various treatment options; or allow before-and-after comparisons of aspects of the region of interest; or to provide quantitative assessments of parameters of interest describing the region of interest from the results.

Systems, processes, and computer-readable media of varying scope are described herein. In addition to the aspects and advantages described in this summary, further aspects and advantages will become apparent by reference to the drawings and by reading the following detailed description.

### BRIEF DESCRIPTION OF THE DRAWINGS

FIG. 1 depicts a simplified block diagram providing a high-level overview of an exemplary embodiment of an iterative vascular analysis system, in accordance with an embodiment of the disclosed subject matter.

FIG. 2 is a block diagram providing a more detailed description of an exemplary embodiment of an input parameter side of the presently-disclosed analysis and modeling system than is offered via the block diagram of FIG. 1, in accordance with an embodiment of the disclosed subject matter.

FIG. 3 is a block diagram showing an exemplary embodiment of an output parameter portion of the presently-disclosed analysis and modeling system in more depth than is offered in the simplified block diagram view of FIG. 1, in accordance with an embodiment of the disclosed subject matter.

FIG. 4 provides an example of showing a centrally-disposed voxel corner point and eight neighboring voxels which are used for template matching, in accordance with an embodiment of the disclosed subject matter.

FIG. 5 illustrates an exemplary fluid mesh sample, in accordance with an embodiment of the disclosed subject matter.

FIG. 6 shows an example of how a model using information relating to a measurement scenario may be augmented, by adding artificial vessel segment models, to usefully employ data obtained from specific measurement locations, in accordance with an embodiment of the disclosed subject matter.

FIG. 7 is a flow chart describing acts in conformance with usage of the disclosed modeling and analysis system, in accordance with an embodiment of the disclosed subject matter.

FIG. 8 is a flow chart describing acts in conformance with an exemplary evaluation protocol employing the disclosed modeling and analysis system, in accordance with an embodiment of the disclosed subject matter.

FIG. 9 is a flow chart describing acts in conformance with an exemplary intra-operative protocol employing the disclosed modeling and analysis system, in accordance with an embodiment of the disclosed subject matter.

FIG. 10 is a flow chart describing acts in conformance with an exemplary post-treatment evaluation protocol employing the disclosed modeling and analysis system, in accordance with an embodiment of the disclosed subject matter.

FIG. 11 illustrates an example of a general computation resource useful in implementation of one or more of the processes of FIGS. 7 though 10 in relation to the system shown and described above with reference to FIGS. 1 through 3, in accordance with an embodiment of the disclosed subject matter.

### DETAILED DESCRIPTION

In the following detailed description, reference is made to the accompanying drawings that form a part hereof, and in which are shown, by way of illustration, specific embodiments that may be practiced. These embodiments are described in sufficient detail to enable those skilled in the art to practice the embodiments, and it is to be understood that other embodiments may be utilized, and that logical, mechanical, electrical and other changes may be made, without departing from the scope of the embodiments.

The detailed description is divided into six sections. In the first section, a system level overview is provided. In the second section, a more detailed discussion of implementation aspects is presented. In the third section, a new mesh model and the application of that new mesh model in the context of the present disclosure is discussed. In the fourth section, processes are described for several different implementations of the techniques and discoveries disclosed herein.

The fifth section discloses hardware and an operating environment, in conjunction with which embodiments may be practiced. The sixth section provides a conclusion which reviews aspects of the subject matter described in the preceding segments of the detailed description. A technical effect of the subject matter described herein includes employing coupled fluid dynamics and mechanical simulation to provide significantly enhanced accuracy information in comparison to a simple fluid dynamics simulation, where the information provided thereby, such as blood flow characteristics and vessel deformation, is important for increased accuracy in treatment planning by enabling richer diagnosis, increased reliability of prognosis of vascular diseases, estimation of the outcome of different treatment methods and determination of appropriate parameters for the selected treatment, such as selection of an appropriate coil or stent type and suitable placement in a user-specifiable region of interest.

Goals of the subject matter disclosed herein include supporting risk assessment, treatment planning, selection of appropriate treatment options in view of presently-available and future treatment modalities and techniques, with a general object of improving treatment and control of vascular diseases. Aspects involved in this process may include performing rupture analysis of the vasculature, modeling hemodynamic effects of different endovascular tools, estimating load-bearing capacity of an aneurysm, or calculating other clinically relevant indicators, including but not limited to parameters such as flow steadiness; average, peak value, gradient of wall shear stress, pressure, displacement, and analogous hemodynamic aspects.

All of these simulations or characterizations utilize detailed information regarding parameters describing a combination of measured and inferred blood flow characteristics, and data relating to time-varying vessel deformation. No generally applicable direct-solution method for measuring blood flow and vessel deformation in vivo is known. Consequently, the disclosed analysis system usefully employs a fluid-structure interaction (FSI) solver, which iteratively employs concatenated computational fluid dynamics and finite element mechanical modeling in order to accurately compute information describing these aspects. The FSI solver may start by employing a combination of presently-available patient-specific data, and tabulated data stored in a database, where the tabulated database includes data entries that correspond appropriately to physical measurements of cadaver-type tissues and other parameters relating to substantially similar scenarios.

The tabulated data entries may be employed in instances where desired aspects of patient-specific measurement results are absent, yet where other, relevant quantities provide information useful and suitable in arriving at appropriate approximations for modeling purposes. This may allow the disclosed tools and techniques to achieve robust support for treatment planning and risk assessment purposes, as is described below in more detail in §I below.

### §I. System Overview

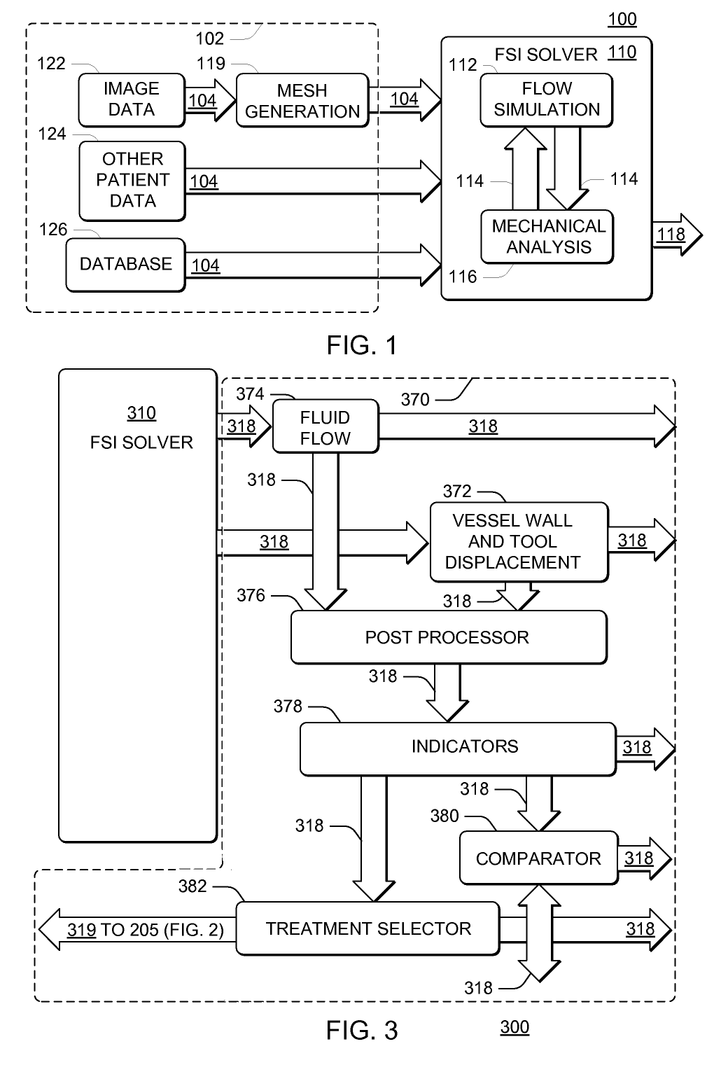

FIG. 1 depicts a simplified block diagram 100 providing a high-level overview of an exemplary embodiment of an iterative vascular analysis system, in accordance with an embodiment of the disclosed subject matter. The block diagram 100 shows a portion 102 of the input side of the system (in dashed outline), with buses 104 interconnecting various elements and coupling the portion 102 to a fluid structure interaction or FSI solver 110, which employs coupled modules for describing the computational fluid dynamics aspects of the blood/fluid flow and a finite element mechanical analysis of the vasculature itself.

The FSI solver 110 includes a flow simulation module 112, which employs computational fluid dynamics to model flow and pulsatile aspects relevant to hemodynamics, buses 114 for coupling data between the flow simulation module 112 and a finite mechanical analysis module 116, and an output bus 118 for communication of raw simulation results to other system components.

The portion 102 includes a number of modules, represented in FIG. 1 as including a mesh generation module 119, an image data importation or lookup module 122, an input module for specifying or accessing other patient data 124 and one or more databases 126, represented here by a single module 126 but which may be realized as multiple organized bodies of data and which may be physically stored in one location or in a variety of locations, depending on the implementation of a specific system 100, as is well known to those of ordinary skill in the art to which the subject matter of this disclosure pertains. In general, the compilations of data represented by the block 126 are accessible to many or all of the elements of the system 100, however, these alternatives and interconnections are not explicitly shown for simplicity of illustration and ease of understanding.

The database 126 may usefully be employed as well for other purposes. Further, the database 126 may be periodically or aperiodically augmented with revised or new information, descriptive of new treatment tools, of additional physical characteristics data via expansion of information obtained, for example, through dissection of relatively inaccessible or other portions of vascular systems, and other types of information. As such, the database 126 typically employs non-volatile read-write memory units for data storage.

When multiple systems 100 share a single database 126, all of those systems 100 benefit from such data augmentation and are kept in data synchronism. The scope for which the information accrued in the database 126 over time may include applications such as are noted the following examples: providing estimates for those parameters that are not available for or could not be acquired for the given patient; comparison of indicators corresponding to examinations performed at different times (e.g., in the context of longitudinal studies); statistical analysis and trending, for example, to determine more and less successful treatment methods for a given problem, and/or to assist in selecting the more relevant indicators; and in calibration assessments such as estimations of reliability of the analysis system 100, etc. Supporting data for such purposes relies strongly on the modeling capabilities provided via the FSI solver 110.

In operation, the FSI solver 110 takes input information from the portion 102 and supplies that to the flow simulation module 112 which is coupled via buses 114 internal to the FSI solver 110 to the mechanical analysis module 116. The flow simulation module 112 computes pulsatile variations of physical properties descriptive of the blood/fluid in a region of interest of vasculature to be modeled, and flow thereof, which initial result is then coupled from an output of the flow simulation module 112 via bus 114 to an input to the finite mechanical analysis module 116.

In turn, that pulsatile loading of the vasculature results in stretching or other physical modulation of the vasculature, which is calculated by the mechanical analysis module 116, responsive to the pulsatile loading as estimated by the flow simulation module 112. The dynamic result of the mechanical analysis module 116 is coupled from an output of the mechanical analysis module 116 back to inputs of the flow simulation module 112 by another bus structure 114. It will be understood that such bus structures 114 may or may not actually correspond to a physically realized bus structure as represented in FIG. 1.

Iterative operation of computation modules 112 and 116 is represented in FIG. 1 by the bus structures 114, and is described below in §IV in more detail with reference to process 700 of FIG. 7. It will be appreciated that such functionality may be realized through other forms of hardware or software, as is well known to those of skill in the relevant arts.

In one embodiment, computer readable code is configured to cause one or more processors to evaluate convergence of concatenated simulations in the FSI solver 110 to an acceptable degree. Results from the concatenated simulations are then supplied to the collection of analysis modules for further processing.

This back and forth, or iterated, calculation process, whereby the distortions of the vasculature are estimated by the mechanical analysis module 116, responsive to pulsatile loading thereof as estimated by the flow simulation module, and the effects which such mechanical distortions in turn impress upon the pulsatile flow as estimated by the flow simulation module 112, etc., proceeds iteratively towards a desired level of convergence.

In practice, this may be determined in any of many ways, such as, by way of example and not intended to be limiting, that a predetermined number of iterations has occurred, or some quantitative measure of convergence, such as a reduction in variation of quantities between successive iterative cycles below some predetermined or user-adjustable threshold, is reached. Other empirically-determined bounds on the iteration process consistent with the quality of results desired may also be employed. When it is determined that convergence has occurred, results are output from the FSI solver via the bus 118.

The kinds of information supplied by the portion 102 to the FSI solver 110 may include: multidimensional data suitable for forming a three-dimensional or four-dimensional image of vasculature geometry in a neighborhood of a region of interest; patient demographic information (patient age, gender, weight, any evidence of abnormalities, such as hypovolemia, or other factors relevant to modeling of properties of the blood/fluid itself, etc.) in order to estimate those parameters desirable for relatively complete analysis but which may not have been measured or possibly cannot be directly measured, specifications descriptive of one or more treatment method definitions, such as defining a region of interest, specification of a menu of tools to be considered for usage and the like, and, optionally, particularly when increased or more accurate patient-specific analysis is desired, additional two-dimensional (2D), three-dimensional (3D) or four-dimensional (4D) image sequences, blood flow and mechanical properties measurement data (a broad variety of other diagnostic data may be utilized, in conformance with the nature of the situation at hand and the judgment of the physician or team involved in the treatment protocol specification and/or implementation.

In order to convey appreciation of the enormous and potentially limitless scope of such information, as well as the seemingly infinite numbers of variations possible, and to demonstrate that an exhaustive listing is neither practical nor desirable in this disclosure, examples of such inputs to the system 100 may include but not necessarily be limited to information describing a three-dimensional aspect of the vasculature, such as voxel data collected via any suitable tool, such as MRI apparatus, fMRI or so-called “functional magnetic resonance imaging” devices and techniques, CAT scanner, X-ray angiography, SPECT or single photon emission compute tomography, ultrasound methodology and apparatus, positron emission tomography, and other modes for collecting information descriptive of blood flow and of vascular conditions and variability responsive to the beating of a heart under resting conditions or under conditions representing exercise. Additionally, information regarding fluid or blood flow, fluid or blood pressure, fluid or blood volume, fluid or blood viscosity and other parameters description of fluid or blood flow and/or vasculature shape and elasticity, fluid or blood leakage and other kinds of information associated with characterization of vasculature performance in vivo and potential for rupture or other undesirable abnormalities may comprise portions of the information useful as inputs to the system 100 or presented or inferable from outputs of the system 100.

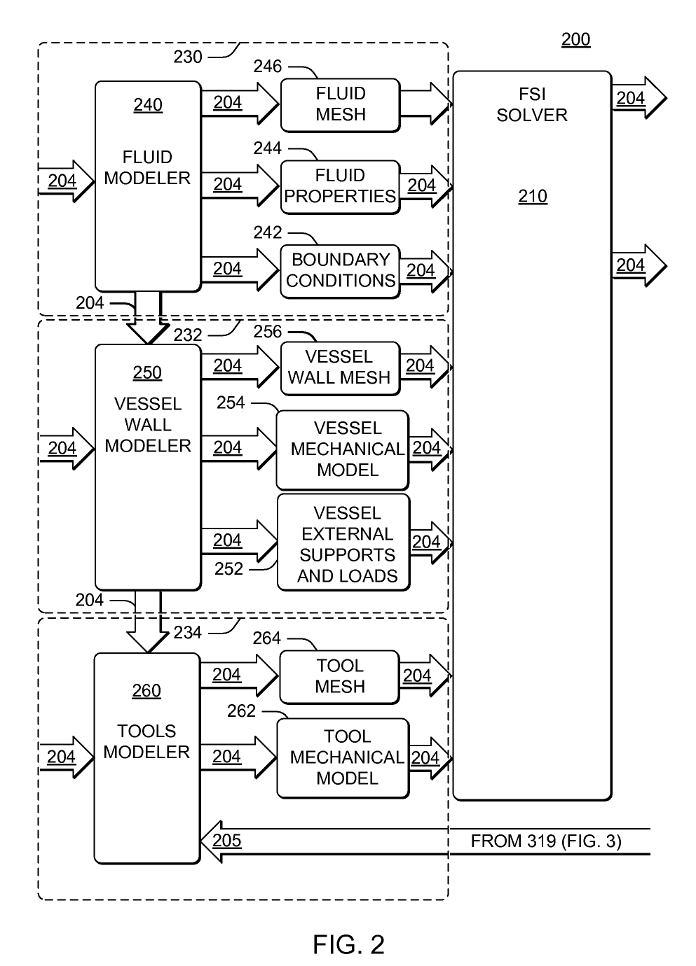

Information including one or more of these kinds of data is often linked to a patient record which may include cumulative data from a series of measurements made at different times, including information describing when such measurements were made and any other sorts of ancillary data typically involved in forming patient records, or measurements made via any of a variety of techniques and measurement tools, together with other information descriptive of the tools, techniques, contrast agents and other relevant data. A more detailed overview of the system 100, coupled with somewhat more complex discussion of the elements and how they interact, follows in the descriptions of FIGS. 2 and 3 in §II below, which should be interpreted in view of the broad-brush overview provided with regard to FIG. 1, supra.

### §II. Implementation Example

FIGS. 2 and 3 provide more detailed block diagrams 200 and 300 of the vascular analysis system 100 of FIG. 1 of the present disclosure, illustrating the input parameters and the modules which employ those parameters to derive a set of data suitable for modeling via the FSI modeling tool 110 of FIG. 1 which is described and taught in the present disclosure, and showing how these elements inter-relate and cooperate in determining data not present in the results of measurements carried out on the subject, and which then is able to automatically or interactively provide stored data presenting a “closest fit” to the presently-available measured information in order to accurately simulate hemodynamic quantities needed for a particular assessment or treatment-planning scenario.

FIG. 2 is a block diagram 200 providing a more detailed description of an exemplary embodiment of an input parameter side of the presently-disclosed analysis and modeling tool than is offered via the block diagram 100 of FIG. 1, in accordance with an embodiment of the disclosed subject matter. In FIG. 2, buses 204 interconnect various elements and portions of the system 200 to an embodiment of a FSI solver 210 (analogous to the FSI solver 110 of FIG. 1; common or analogous features in different illustrations are frequently referenced by the portion of the reference character sequence following the initial descriptor indicative of the specific figure involved). Other major sub-systems such as a fluid-modeling module 230, a vessel wall modeling module 232 and a tool-modeling module 234 each accept inputs, such as measured data relative to the patient or analogous information as provided via the database 126 of FIG. 1 (which is coupled to all relevant elements, although such interconnections are not explicitly shown, for simplicity of illustration and ease of understanding), and provide outputs which are in turn coupled to inputs to the FSI solver 210.

The fluid-modeling module 230 includes a fluid modeler 240, and sub-sub modules such as a boundary conditions calculator 242, a fluid properties calculator 244 and a fluid mesh generator 246, each having inputs coupled via buses 204 to the fluid modeler 240. These each have outputs coupled to the FSI solver 210 via additional buses 204. The fluid-modeling module 230 generates the blood, artificial blood or other fluid flow-related inputs (blood mesh, blood fluid properties and blood flow boundary conditions) to the FSI solver 210.

The fluid dynamics simulation requires accurate description of the flow, at least at the boundaries of the mesh employed to model the blood or other fluid. On boundary mesh elements that have common surface with the vessel wall, no slip, and a hydraulically smooth wall is assumed. Blood flow for inlet and outlet mesh elements can be defined by mass flow rate (or equivalently velocity or volumetric flow rate, pressure) function in time.

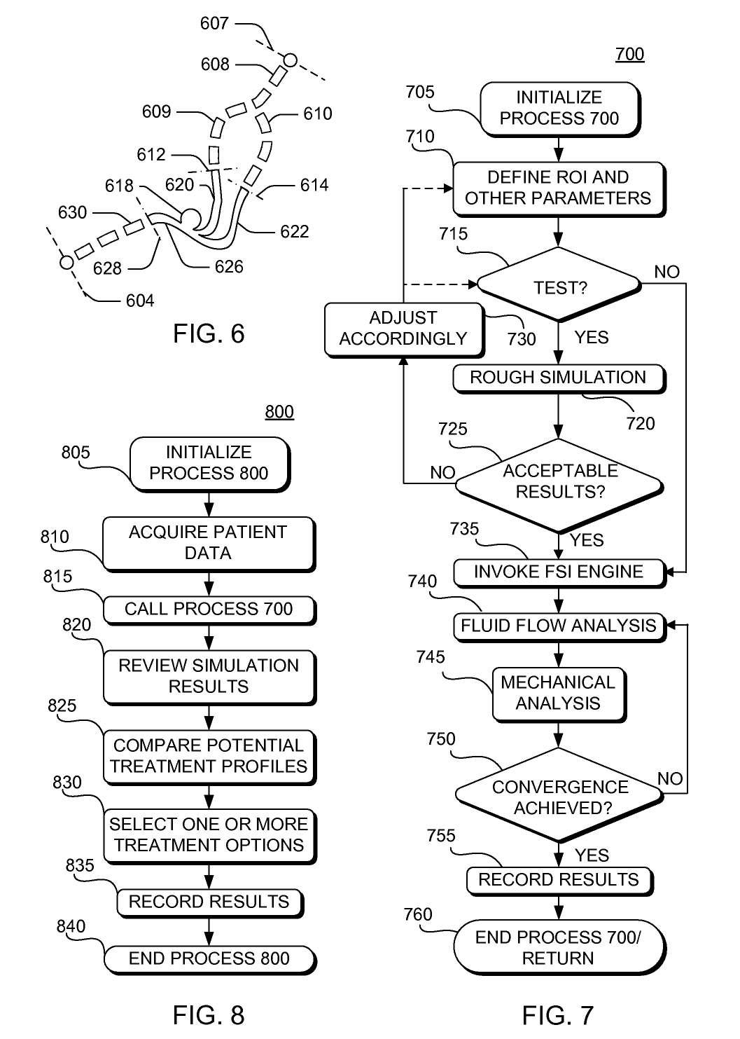

There are two primary options for the determination of the mass flow rate function in time. A first option includes direct measurement, where three-dimensional flow is directly measured at multiple time instances (e.g., by MRI). It is advisable to define the function not only in the inflow and outflow, but in as many regions as possible. Alternatively, in a second option, indirect measurement is employed. When no full three-dimensional measurement in time is available or is not available right at the inflow and outflow the flow (e.g., 4D CT, ultrasound or blood pressure measurements), then additional artificial vessel segments are appended to the model (see FIG. 6, infra) of the region of interest in order to simulate vasculature between the location of the measurement and the model.

All measurements are stored in the database 126 of FIG. 1, so that when there is no available measurement data, or when just a few parameters can be determined (e.g., mean blood pressure), then flow rate function of a similar patient can be used.

The vessel wall modeling module 232 includes a vessel wall modeler 250, coupled via buses 204 to sub-sub modules, such as vessel external supports and loads calculator 252, a vessel mechanical model calculator 254 and a vessel wall mesh generator 256. These each have outputs coupled to the FSI solver 210 via additional buses 204. The vessel wall modeler 250 generates the vessel-wall-related inputs (vessel wall mesh, mechanical model, material properties and external supports) to the FSI solver 210.

In addition to the forces induced by the flow of blood or other fluids, the tissues surrounding the vessel wall also have an influence on the deformation. The vessel external supports and loads calculator 252 in the vessel wall modeler 250 calculates the latter effect by applying three-dimensional elastic supports around the external wall of the vasculature. The parameters of the supports corresponding to the current patient are retrieved from the database. In addition to this uniform support, additional local constraints can be defined by the user, or automatically. Local constraints can be determined from the diagnostic image, and/or by analyzing the neighborhood of the vasculature (when it is close to a bone surface or other non-elastic structure then a local constraint shall be added).

The vessel mechanical model calculator 254 in the vessel wall modeling module 232 uses the conventional Mooney-Rivlin material model to describe the non-linear mechanical properties of the vessel wall. It will be appreciated that other material models may be alternatively employed, including but not limited to the conventional Ogden model. The model parameters are determined by measurements of dissected vasculature tissues, and the measurement results are stored in the database 126 of FIG. 1. The data that is the most similar to the observed vessel segment is retrieved from the database and used for the analysis. The model parameters in one vessel segment can differ from the parameters of another segment (depending on vessel type, size, calcification, etc.) and the same segment may be a composite of multiple materials.

The vessel wall mesh generator 256 coupled to the vessel wall modeler 250 operates in coordination with the blood mesh generator 245. Vessel wall thickness (and this varies along the vasculature) is a required parameter for this process. Vessel wall thickness can be determined in one of the following ways: (i) a direct measurement via three-dimensional characterization or intravascular ultrasound may be performed and employed; and/or (ii) indirect measurement can be done by measuring actual deformation of the vasculature due to blood pressure change within the cardiac cycle. This can be measured by high temporal resolution imaging modalities (e.g., X-ray fluoroscopy, ultrasound), or deformation between two time instances when the mean blood pressure is different can be measured by a high spatial resolution imaging modality. The wall thickness (and potentially other physiological parameters) can be determined based on the deformation information, the blood flow induced forces acting on the wall and the wall material model.

A third approach is to estimate wall thickness by retrieval of similar data from the database 126 of FIG. 1. When direct measurement is not available, then a database is used to estimate the wall thickness. The database 126 is built from measurements of dissected vasculature tissues (e.g., healthy vessel of different sizes at different places, aneurysm wall thickness of different parts of the aneurysm). The thickness is determined by selecting the data that is most alike the current vasculature.

The tool-modeling module 234 includes buses 204 coupling a tools modeler 260 to each of a tool mechanical model calculator 262 and a tool mesh generator 264. The tool-modeling module 234 also accepts data via a bus 205, as will be described below in more detail with reference to FIG. 3. Common aspects of the fluid mesh generator 246, vessel wall mesh generator 256 and tool mesh generator 264 will be described below in more detail with reference to §III.

The tools modeler 260 benefits from the fact that most of the tools that are used during vascular interventions already have a mechanical model (including mesh and material properties). As a result, during the treatment definition or optimization, the user specifies the size and position and other additional parameters of the tools, and a corresponding model and related data are extracted or recalled from the database 126 of FIG. 1. All of this information is then sent to the FSI solver 210.

Operation of the FSI solver 210 was discussed above with regard to FIG. 1. However, in general, there are many commercially available solvers for finite element analysis. The requirement for the FSI solver 110, 210 to be used in the vascular analysis system is 100, 200 that it has to support the solution of two-way coupled fluid structure interaction for the material model and mesh types that are provided by the blood, vessel wall and tools modelers.

Before starting the full simulation a simplified solution is optionally generated (assuming rigid vessel wall and other simplification in the material model and simulation parameters). This gives just an approximate result, but such an approximation facilitates a quick verification of proper problem definition, prior to invoking the more time-consuming and resource-intensive full computation.

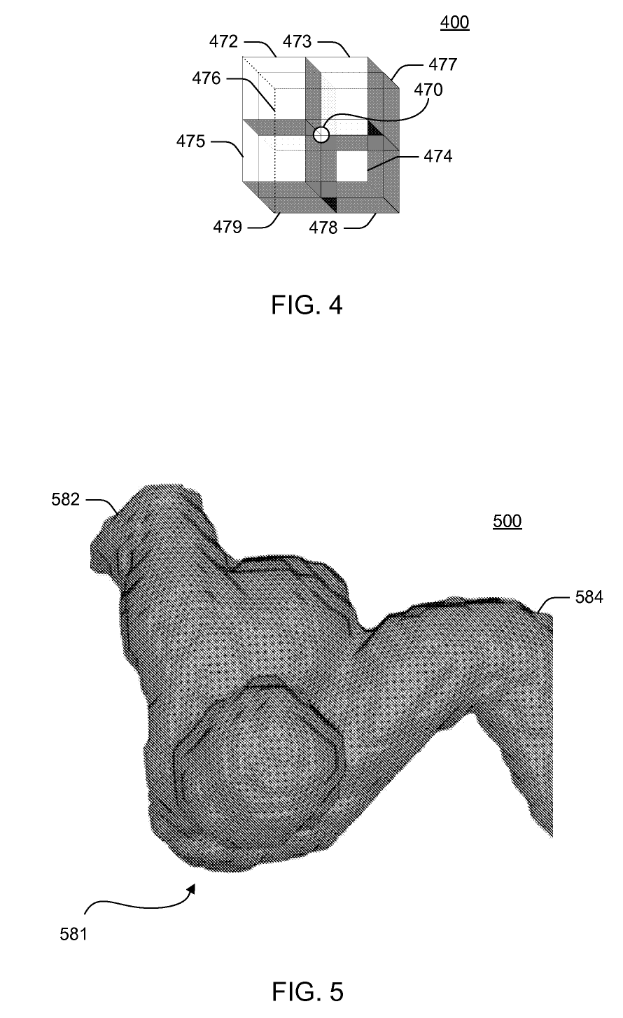

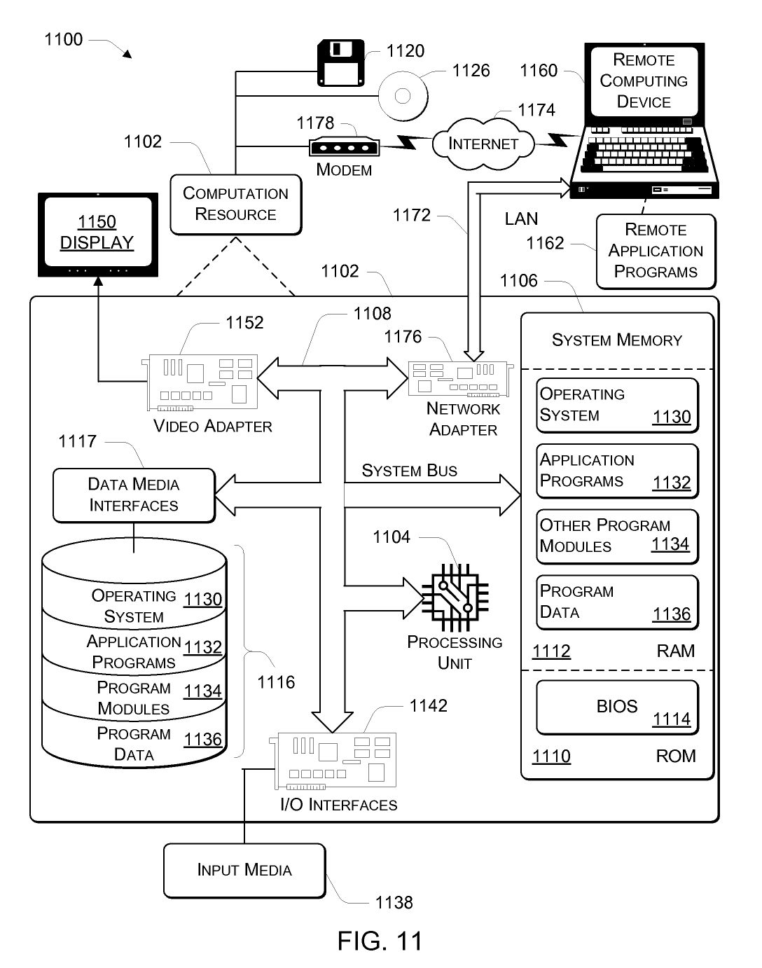

The problem definition and the display and post-processing of results can be performed optimally on an average workstation. However, a fast enough computation of the full FSI simulation requires high computing performance. The FSI solver module 110, 210 may use the services of a remote computation server to achieve this, as is described below with regard to FIG. 11, among other places. Examples of suitable software for such computation include the Ansys Multiphysics package, available from ANSYS, Inc. (ansys.com/products/default.asp) (leading portions of the URL have been omitted in order to avoid problems encountered by unsophisticated parties). An example computation of an FSI problem, using a mechanical model consisting of 12,000 nodes (see, e.g., FIGS. 4 and 5 in §III, infra) and a fluid model of 65,000 nodes, requires about six hours on a Core 2 Duo Q6600 3.2 gigaHertz machine. The performance of this machine is 2973.23 million operations per second or 2.97 gigaflops, as measured by Distributed Computing program Einstein(@)Home (parentheses added to preclude inadvertent browser-launching errors by unsophisticated parties).

Actual implementation of a conventional FSI computation engine 110, 210 is complex, and may differ from the present description in a variety of ways, as is known to those of skill in the relevant arts. For example, the FSI solver 110, 210 may initiate by invoking either fluidic or mechanical analysis, or the mechanical and fluid analyses may run in parallel, etc.). In this application, the fact that information and results from each of these analyses are employed in the other analysis approach during the course of iteration of the computations represents departure from conventional methodologies, particularly with reference to the field of application of the subject matter of the present disclosure.

The remote computation server can receive problem definitions from multiple workstations through network connections, quickly perform the resource-intensive computations and send back the raw computation results to the workstation. A computation server can be shared among multiple workstations in the same institution, or shared between multiple institutions. Outputs from the FSI solver 210 are conditioned further to provide a variety of different outputs, depending on the nature of the overall task at hand, as is described below in more detail with reference to FIG. 3.

FIG. 3 is a block diagram 300 showing an exemplary embodiment of an output parameter portion of the presently-disclosed analysis and modeling modules in more depth than is offered in the simplified block diagram view 100 of FIG. 1, in accordance with an embodiment of the disclosed subject matter. In FIG. 3, the block diagram 300 shows a FSI solver 310 providing outputs via buses 318 to a variety of analysis modules.

The analysis modules include a vessel wall and tool displacement module 372 and a fluid flow module 374 which also each provide outputs to further system elements via buses 318. A post-processor 376 accepts inputs from the fluid flow module 374 via a bus 318 and from the vessel wall and tool displacement module 372 via another bus 318 and supplies output signals to a module of indicators 378 via another bus 318. The post-processor 376 computes derived quantities and various indicators from the raw simulation results (displacements, velocity and pressure fields), which are then displayed to the user and/or further analyzed.

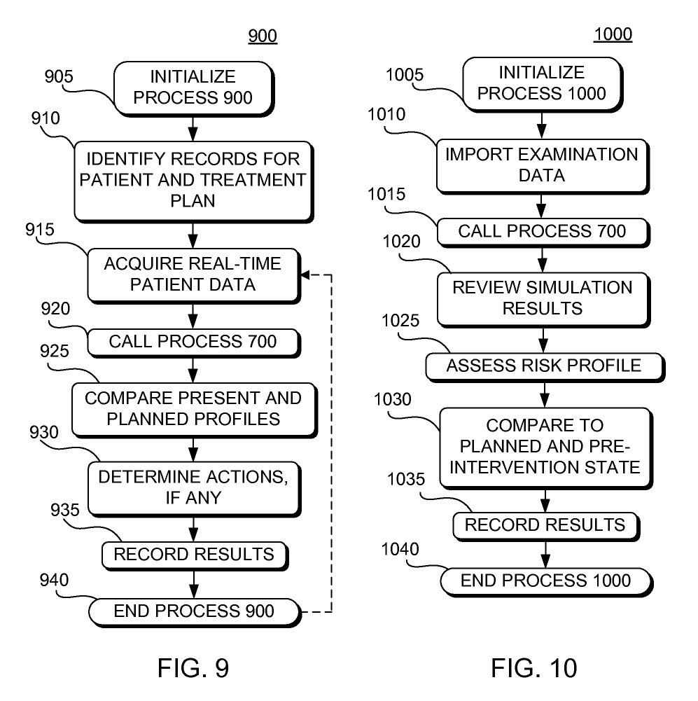

A comparator 380 accepts input signals via buses 318 and supplies output signals via another bus 318. The comparator 380 is used in analyses where two or more sets of outputs are being compared, such as with regard to the intra-operative process 900 of FIG. 9, and the post-operative process 1000 of FIG. 10, respectively, as described in more detail below in §IV.

A treatment selector module 382 is coupled to the indicators module 378 via a bus 318 and has one output coupled to further system elements via another bus 318 and sends data back to the input sections of FIG. 2 via a bus 319 which couples to the bus 205. This may permit a selected treatment option to be analyzed in more detail. The treatment selector module 382 determines treatment parameters (treatment methods, parameters, positions of tools, etc.) that lead to preferred indicator values (minimum risk or rupture, minimum shear stress, maximum occlusion, minimum displacement amplitude in aneurysm, etc.) and hence result in facilitate in treatment selection, as is described below in more detail in §IV with reference to flow chart 800 of FIG. 8.

By now it may be appreciated that the system 100 of FIG. 1, as described in more detail with reference to FIGS. 2 and 3, is able to address a broad variety of tasks through accurate, robust and rapid modeling of vascular scenarios. Examples illustrating the richness of output data from the system may encompass patient-specific information including at least: three or four dimensional display of blood/fluid flow and structural information about the vasculature in any region or regions of interest, such as flow patterns, wall displacements, etc., for purposes such as qualitative visual assessment, at different time steps throughout a cardiac cycle; display of various indicators, such as hemodynamic aspects including flow steadiness, average, peak value, gradient of wall shear stress, pressure, occlusion, etc.; mechanical aspects including such elements as displacement amplitude, Von Mises stress, etc.; and aiding in deriving recommendations for treatment planning (described in more detail in §IV below with respect to flowchart 800 of FIG. 8), for example, preferred sizes, placements, and suitable parameters of tools and devices used in treatment; and, clearly, comparison all the above information before, during and after treatment.

Thus, to briefly recapitulate, vessel deformation affects blood flow, and vice-versa. As a result, flow-induced loads are recomputed in order to provide more realistic and accurate results. In turn, those results are employed to derive revised estimates of vessel deformation, and, in the disclosed subject matter, iteration of such calculations is employed to rapidly derive robust estimates which account for the interactions of the coupled flow and mechanical aspects of vessel functionality.

In order to calculate effects due to pulsatile loading of vessel deformation, blood-flow-induced loads acting on the vessel wall are determined. Then, resultant vessel deformation is estimated via computation. In order to accomplish that efficiently in the context disclosed herein, a new methodology and modeling approach was developed. In this approach to volumetric mesh generation for finite element mechanical analysis, the main input of the system is the three-dimensional image of the vasculature. From that, a geometric model (viz., a volumetric mesh) is generated. Other parameters (boundary conditions, material properties, etc.), for example, those which are generally quite significant for the analysis, may be determined from patient specific measurements, or may be retrieved from the database 126, predicated on correlation with patient-specific information, where applicable.

The system 100 also includes memory devices (not explicitly shown in FIGS. 1 through 3), coupled via the buses 104 to elements of system 100 through suitable interfaces. The database 126 is one example of stored data desirably embodied in a non-volatile and possibly read-write memory, which may be a part of the system 100 or which may be included as a remote element couplable to the system 100, as noted in more detail below with reference to FIG. 11.

Memory devices providing non-volatile read-write capabilities are usefully employed to store patient information, records of various measurements, and software tools for analysis of such data and for formatting such information for display via a conventional monitor or other devices (not explicitly shown in FIGS. 1 through 3). Memory devices also find utility in for storing one or more databases containing parameters descriptive of vessel characteristics, of the various kinds of tools available for treatment of vascular illness or abnormality, and the like, and the databases containing such kinds of information are accessible to the various system elements shown in FIGS. 1 through 3, although illustration of such conventional interconnections has been omitted from those FIGs. in order to promote clarity of illustration and for ease of understanding.

Datasets representing four-dimensional (e.g., with time as a fourth dimension, in addition to the conventional three spatial dimensions, in other words, representing information analogous to a movie or other dynamic record of vascular system performance), three-dimensional data and image or two-dimensional data (i.e., data in pixel form or analogous representation schemes) typically conform to the digital imaging and communications in medicine (DICOM) standard, which is widely adopted for handling, storing, printing, and transmitting information in medical imaging. The DICOM standard includes a file format definition and a network communications protocol. The communication protocol is an application protocol that uses TCP/IP to communicate between systems. DICOM files can be stored in memory devices and retrieved therefrom, and may be exchanged between two entities that are capable of receiving image and patient data in DICOM format, for example via a network.

The memory devices include mass data storage capabilities and one or more removable data storage device ports, as is described later in more detail with reference to FIG. 11. The one or more removable data storage device ports are adapted to detachably couple to portable data memories, which may include optical, magnetic and/or semiconductor memories and may have read and/or write capabilities, and which may be volatile or non-volatile devices or may include a combination of the preceding capabilities.

### §III. Mesh Model

The most important patient-specific parameter is the volumetric mesh used to simulate the blood or fluid properties that is used for computational fluid dynamics (CFD) analysis. The mesh consists of thousands of basic geometric elements defined by points and connections between them. The mesh can be constructed from an image or equivalent data of any modality, which can capture the three-dimensional geometry of the vasculature lumen (typically contrasted three-dimensional X ray angiography, CT or MRI volume).

Although there are several methods for creating a volumetric mesh from an image volume or equivalent data, the present disclosure teaches a new method, having the following characteristics: (i) it is very simple, fast and robust; (ii) it generates tetrahedral mesh directly from the volume image/data at acceptable quality for FSI analysis by the FSI modeler 110, 210, 310 of FIGS. 1 through 3, respectively; and (iii) it generates both the blood and the vessel wall mesh with common node elements at the boundary surface (which favors efficient FSI solution). The blood mesh is generated in the fluid mesh calculator 246 (FIG. 2) by iterating through all the corners of blood voxels in the volume and matching a template to all the voxels that touch that specific voxel (a total of 8 voxels, see FIG. 4).

As a pixel set consists of 8 voxels, and a voxel can have two possible values (blood or non-blood), there are altogether 256 possible templates. The template defines how many tetrahedron elements shall be added to the mesh for the given set of voxels and in what configuration. It works very similarly to the conventional and widely-used marching cubes algorithm. The main difference is that this new algorithm creates a volumetric mesh, which can be used for FEM analysis directly. The surface of an example of a resulting blood mesh is shown in FIG. 5.

For the vessel wall mesh generation by the vessel wall mesh calculator 256 (FIG. 2), the original image is modified by applying dilation on the blood voxels (by the thickness of vessel wall) and then the voxels corresponding to the blood mesh are removed. It also uses the same template-based meshing on the modified image that was used for the blood mesh. The templates are designed to be invertible, so that when the blood mesh elements are removed the internal surface of the blood mesh is perfectly aligned to the outer surface of the blood mesh (they have common node points, which facilitates an efficient numerical solution).

FIG. 4 provides an example 400 of showing a centrally-disposed voxel 470 corner point and eight neighboring voxels 472, 473, 474, 475, 476, 477, 478, 479, which are used for template matching, in accordance with an embodiment of the disclosed subject matter. Starting from the upper left-hand corner, the voxel 472 is part of a first or top layer of voxels which comprise a face of a cubic shape of the example 400 that is closest to the viewer, and, proceeding clockwise, a remaining three of the four total voxels forming that face are voxels 473 (upper right-hand corner), 474 (lower right-hand corner) and 475 (lower left-hand corner). A rearward face of the cubic shape is formed, again starting from a portion adjacent the upper left-hand corner, via a voxel 476, and, proceeding clockwise, remaining voxels comprising that portion of the cubic shape 400 are voxels 477, 478 and 479.

A blood/fluid mesh is generated, corresponding to the operations associated with the mesh generation module 106 of FIG. 1, and the fluid mesh generation module 246 of FIG. 2, by iterating through all corners/vertices, e.g., analogous to the corner 470 illustrated above, of blood or fluid voxels in the volume being modeled, and matching a template to all of the eight voxels (as shown in FIG. 4) touching that specific voxel corner. For the present purpose, a voxel, such as any of the voxels 472 through 479, may have one of two possible values (blood/fluid or non-blood/non-fluid), and, accordingly, there are altogether two raised to the power of eight, or two hundred and fifty-six, possible different templates.

For the vessel wall mesh generation, the original image data, or information from which that may be constructed, is modified by applying dilation (or the equivalent thereof) on the blood/fluid voxels, magnifying them by an amount given by the thickness of vessel wall. As a result, those voxels corresponding to the blood/fluid mesh are removed. This operation is followed by the same template-based meshing on the modified image data that was used for the blood/fluid mesh generation. The templates are designed to be invertible, so that when the blood/fluid mesh elements are removed, the internal surface of the blood/fluid mesh is fully aligned to an outer surface of the blood/fluid mesh. A consequence of the above-noted procedure is that they have common node points, which facilitates efficient numerical solution.

In the computations associated with the fluidic physical properties module 244 in FIG. 2, appropriate blood/fluid physical properties are retrieved from the database, based on the patient demographics data indexed through operation of the patient data module 124 of FIG. 1. The database 126 includes a substantially complete set of actual measurements of such blood/fluid properties, spanning a full range over which such parameters vary in practice. For the simulations to conform to Newtonian fluidic behavior (e.g., viscosity is not a function of pressure in Newtonian fluids), constant viscosity and density for the blood/fluid are assumed.

FIG. 5 illustrates an exemplary fluid mesh sample 500, in accordance with an embodiment of the disclosed subject matter. The exemplary mesh sample 500 includes a region of anomalous or diseased vasculature 581 that is part of the region of interest, as well as a first port 582 and a second port 584, each corresponding to relatively normal vasculature and disposed at either end of the anomalous or diseased vasculature portion 581 to be modeled. The first 582 and second 584 ports correspond to the inlet and outlet (or vice versa) for the anomalous or diseased vasculature portion 581, with all of the blood/fluid that passes through one of the first 582 or second 584 ports also passing through the corresponding other of the second 584 or first 582 ports. The example 500 of FIG. 5 may represent what in actuality is more than one vessel (such as furcations associated with progressively finer vasculature, ultimately supplying blood/fluid to capillary structures), as is described below in somewhat more detail with reference to FIG. 6.

FIG. 6 illustrates an example 600 of a model of a region of interest having a first input measurement plane 604 (analogous to either the first port 582 or the second port 584 of FIG. 5) and a second measurement locus 607 (analogous to either the second port 584 or the first port 582 of FIG. 5). Artificial vessel segments 608 and 609, 610 accommodate a furcation in the vessel being modeled, and planes 612, 614 illustrate where those artificial model segments join to an aneurism 618 via blood vessel segments 620, 622. An additional blood vessel segment 626 couples another end of the aneurysm 618 in the vessel being modeled to a plane 628 that in turn is coupled via artificial model segments 630 to join the vessel with the first measurement locus 604.

FIG. 6 shows an example 600 illustrating how information relating to a measurement scenario may be augmented, using artificial vessel segment models 605, 608, 609, 610, to usefully employ data obtained from specific measurement locations, in accordance with an embodiment of the disclosed subject matter. This permits more accurate modeling of a vessel segment when the segment itself cannot be directly measured, and is being modeled via data taken from a dissected specimen, for example. Aspects of the measurement processes, problems and analysis in several different contexts are discussed below with reference to §IV.

### §IV. Processes

In the following section, some exemplary processes are described with reference to FIGS. 7 through 10 in the context of measurements corresponding to various phases of patient assessment and treatment. These include pre-operative characterization and treatment planning, intra-operative monitoring and post-operative follow-up and monitoring. A first aspect of these processes is described below with reference to FIG. 7, which describes generalized operation of the FSI solver which is common to each of these phases of patient treatment.

FIG. 7 is a flow chart 700 describing acts in conformance with usage of the disclosed modeling and analysis modules, in accordance with an embodiment of the disclosed subject matter. The process 700 begins in a block 705.

In the block 705, data may be assembled and input to the FSI solver. Elements of data needed in order to complete an analysis, but which are not present in the results of measurements performed on the patient, may be supplied from the database of representative vascular data, by selection of parameters in conformance with the data to be analyzed. Control then passes to a block 710.

In the block 710, a region of interest and parameters associated therewith are defined. Control then passes to a query task 715.

In the query task 715, a user is asked if there is desire to perform a limited, quick evaluation of the characteristics of various types and conditions of vascular segments in the context of a user-defined vascular region of interest and associated environments, as well as verification of suitable range of tools via the properties of tools associated with treatment of vascular abnormalities, and any patient-related indicia, or information identifying such, associated with the task at hand.

When the user indicates that there is desire to perform a limited, quick evaluation, in order to confirm that the correct information is present and that the region of interest is appropriately defined, control passes to a block 720.

In the block 720, a rough simulation, which does not involve the detailed FSI solver 110, 210, 310 (FIGS. 1 through 3, respectively) operation, but instead utilizes a highly simplified model, such as one which assumes rigid vessel walls, and other simplifications in the material model and simulation parameters. This gives an approximate result, useful for quick verification of appropriate problem definition, and allows for adjustment when the problem definition appears to require refinement, prior to invoking the more time consuming and resource-intensive full FSI-solver computation. Control then passes to a query task 725.

In the query task 725, the user has opportunity to determine that the region of interest appears to be correctly identified, and that the information being presented conforms to what is expected from a rough estimation of the scenario at hand. When the query task 725 determines that something appears to be awry with the problem definition, control passes to a block 730.

In the block 730, adjustments are made in conformance with the irregularities noted by the operator or user, and control then reverts to either the block 710, when the region of interest and similar information appears to be inappropriate specified, and from there to the query task 715, or passes directly to the query task 715, as appropriate, and the sequence resumes as described.

When the response determined by the query task 715 does not indicated need or desire for a rough estimate, or when the query task 725 determines that the results of the rough simulation were acceptable, control passes to a block 735.

In the block 735, the FSI engine or solver (i.e., as shown at 110 in FIG. 1, 210 in FIG. 2 and 310 in FIG. 3) is invoked. The FSI engine (110, 210, 310) then initiates the fluid flow analysis (see, e.g., block 112, FIG. 1) in a block 740, as described supra with reference to FIGS. 1 through 3, and control passes to a block 745, where mechanical analysis (as described above, for example, with reference to block 116, FIG. 1) of the vasculature throughout the region of interest as defined above in the block 710 is performed, in light of the results obtained from the fluid flow analysis of the block 740. Control then passes to a query task 750, or the processes of the blocks 740 and 745 may be iterated a predetermined or user-determined number of times (which may be set in the course of the problem definition phase associated with the blocks 705 and 710), prior to control passing to the query task 750.

In the query task 750, conventional convergence testing is performed. As noted previously, any of a variety of criteria may be employed, and either pre-set criteria may be used to determine an acceptable degree of convergence, a user may select from a menu of such pre-determined set-points, or a user may determine both the manner in which convergence is tested and thresholds relative to that act. Irrespective of how that is handled, a “backup” test determines if or when the process 700 is failing to converge and a suitable error signal and possibly some diagnostic criteria are generated and made available to the user. When the query task 750 determines that convergence is not satisfactory, control reverts to the fluid flow analysis of the block 740, and this proceeds from the juncture at which the query task 750 was invoked. When the query task 750 determines that convergence is satisfactory, control passes to a block 755.

In the block 755, the results from the process 700 are recorded. Generally, these may be recorded in a storage media accessible to the system 100 of FIG. 1, 200 of FIG. 2 and 300 of FIG. 3, and may also be recorded in storage media accessible to the FSI solver or engine 110, 210, 310. Control then passes to a block 760.

In the block 760, control returns to the process (e.g., as described with reference to FIGS. 8 through 10, infra) which called the process 700. The process 700 then ends.

FIG. 8 is a flow chart 800 describing acts in conformance with an exemplary evaluation protocol employing the disclosed modeling and analysis modules, in accordance with an embodiment of the disclosed subject matter. The process described with reference to FIG. 8 is appropriate at least in situations where an aneurism is being detected or investigated for treatment after initial detection. After the detection of an aneurysm, the analysis system can be used before, during and after the treatment.

In a pre-operative context, the sequence of acts might follow as described below with reference to FIG. 8. The process 800 of FIG. 8 initiates in a block 805.

In the block 805, the process 800 is initialized. In one embodiment, initialization of the process 800 includes acts such as entry or importation of patient demographics information. Control then passes to a block 810.

In the block 810, appropriate available diagnostic data (e.g., three-dimensional descriptive data or images, four-dimensional descriptive data or images, such as time sequences of spatial descriptions, relevant flow measurements and the like) may be invoked, measured or recalled from prior assessment results stored via the database (e.g., the database 126 as described above with reference to FIG. 1).

Also, optionally, in the block 810, treatment approaches to be analyzed may be selected, for example via definition treatment method(s) which are supported by available tools, or which are consistent with tools which have been selected for use or for consideration for usage. Parameters such as placement of such tools vis-à-vis the region of interest, and other suitable and/or allied types of information may be added or adjusted in the block 810.

In some instantiations, the acts associated with the block 810 may include definition of a region of interest, or the definition of such may benefit from refinement. Control then passes to a block 815.

In the block 815, the process 700 of FIG. 7 is invoked. Following return 760 from the process 700, control will be passed to a block 820.

In the block 820, results from the FSI solver are reviewed. As noted above with regard to the query tasks 715 and 725 and other associated aspects of the process 700, review of a rough estimate, or of a full simulation, may suggest benefit to adjustment of boundary conditions, “tweaking” or adjustment of aspects affecting the defined region of interest, or modification of one or more of the other simulation data inputs, or evaluation of the sensitivity of desired results to various parameters may be desirable. When those aspects have been resolved satisfactorily, control passes to a block 825.

In the block 825, potential treatment profiles and anticipated results of specific treatments may be compared, based on results for each anticipated potential venue being evaluated. Strengths or weaknesses of one treatment approach or another may be flagged as having particular or dispositive significance with regard to various of the treatment options under contemplation at the time. Control then passes to a block 830.

In the block 830, one or more treatment options may be selected for further consideration, or a particular treatment option may be determined to be preferred, and/or one or more treatment possibilities may be deferred from further consideration and study at this time. Control then passes to a block 835.

In the block 835, results of the adjustments and selection processes and comparisons of various potential alternatives are recorded. For example, such results may be stored in a patient records portion of the database 126 described above, and/or may be exported to other types of resources, along with a preferred treatment plan, if such has been selected. Control then passes to a block 835, and the process 800 terminates.

FIG. 9 is a flow chart 900 describing acts in conformance with an exemplary intra-operative protocol employing the disclosed modeling and analysis modules, in accordance with an embodiment of the disclosed subject matter. The process 900 begins in a block 905.

In the block 905, the process 900 is initialized. In other words, the patient is identified. Control then passes to a block 910.

In the block 910, data descriptive of a region of interest which has been previously determined is recalled from storage, or is imported from other resources. Also, in the block 910, a treatment plan is identified among records associated with the identified patient, and which has been previously selected for this patient is identified in the records, along with identification of results from the previous analysis. These selections may either be determined by an operator, or may automatically be identified using stored information derived from a prior analysis and selection, note of which previously had been stored together with the other patient information. In either event, the results of that prior analysis are brought forward. Control then passes to a block 915.

In the block 915, real-time images are acquired which are relevant to the region of interest. These real-time images, and results from any other appropriate measurements which are contemporaneously performed with the acquisition of real-time descriptive information are collectively transferred to the input portions (such as the portion 102 of FIG. 1 of the system 100, or analogous aspects of the system 200 of FIG. 2, for example). Control then passes (transparently, with regard to the operator or physician) to a block 920.

In the block 920, the process 700 of FIG. 7 is invoked, passing the contemporaneous information gleaned with regard to the block 915 above to the FSI solver 110 of FIG. 1 or 210 of FIG. 2. Control then passes to a block 925.

In the block 925, the present profile, resulting from analysis of the information derived via the acts noted above with regard to the block 915, is compared to the analysis of the previously-selected scenario as determined above in conjunction with the block 910. Anomalies are noted, as well as congruencies and suitable similarities with anticipated or hoped-for results. Control then passes to a block 930.

In the block 930, any actions which are deemed appropriate, based on informed comparison of the presently-achieved scenario, and the previously-designated preferred plan profile, are implemented. Control then passes to a block 935.

In the block 935, information derived from the comparisons, as well as any actions determined to be appropriate, as well as the anticipated or measured influences manifested in conformance with any actions determined to be appropriate in the block 930, are recorded, and/or exported, as has been described supra with regard to the block 830 of FIG. 8, for example. Control then passes to a block 940, and the process 900 terminates.

In some embodiments, a practical aspect of the termination noted at the block 940 is actually to continuously re-iterate those aspects of the process 900 from, for example, block 915 forward, to realize a continuously-updated real-time observational tool for tracking process during a procedure, as indicated by the dashed arrow extending from the block 940 up to and pointing toward the block 915. This may continue until such time as an affirmative “STOP” command is input from a user console, or is otherwise effectuated (for example, when disconnection of probes or other measurement tools from the patient results in affirmative “NO GO” signals being automatically generated within the system 100 of FIG. 1, or analogous other representations).

Optionally, in conjunction with the tasks associated with the block 925, information (such as, by way of example, fluid flow patterns, tool position, degree of occlusion, etc.) may be superposed atop the live image, and may be correctly registered therewith, as an overlay, or may be displayed in a separate view. As well, geometrical and flow information may be gleaned or retrieved from the live images (although they maybe incomplete and of limited accuracy). Using such information, the pre-operative model definition may be updated. Optionally, a quick simulation (e.g., as described above with reference to the block 720 of FIG. 7) may be performed in order to compute indicators and to assist in deriving a modified treatment plan.

FIG. 10 is a flow chart 1000 describing acts in conformance with an exemplary post-treatment evaluation protocol employing the disclosed modeling and analysis modules, in accordance with an embodiment of the disclosed subject matter. Such follow-up is highly desirable, at least in part because recurrent aneurysms can be due to coil compaction or migration or dislocation. Also, in some cases, a de novo basilar tip aneurysm may develop within a few months after treatment via clipping, for example. When such events occur after treatment of a rupture, the probability of fatality in the event of re-rupture is quite high. The process 1000 begins in a block 1005.

In the block 1005, the process 1000 is initialized, by providing indicia identifying the patient. Those indicia are used to identify and extract data from prior measurements of the region of interest, as described above with reference to the database 126 of FIG. 1. Control then passes to a block 1010.

In the block 1010, data from a present examination of this patient are imported into the system 100. Control then passes to a block 1015.

In the block 1015, the process 700 is invoked, to process the data collected in the block 1010. Control then passes to a block 1020.

In the block 1020, results from the simulation derived from the process 700, using the contemporary data collected in the block 1010, are reviewed. Control then passes to a block 1025.

In the block 1025, a presently-applicable risk profile is derived from the results from the simulation of the block 1015 is developed. Control then passes to a block 1030.

In the block 1030, the risk profile developed in the block 1025 is compared to the planned results and risk profile, and to the pre-intervention state data for the patient, as retrieved in the block 1005. Control then passes to a block 1035.

In the block 1035, results from the preceding blocks are integrated into the patient record and are stored in the database 126 of FIG. 1, and/or are exported to other resources. Generally, these results may be recorded in a storage media accessible to the system 100 of FIG. 1, 200 of FIG. 2 and 300 of FIG. 3, and may also be recorded in storage media accessible to the FSI solver or engine 110, 210, 310. Control then passes to a block 1040, and the process 1000 ends.

It will be appreciated that in order to determine outputs with robustness, repeatability and relevance, the system 100 functions at qualitatively better levels in conformance with increasingly precise descriptions of the blood/fluid flow in vasculature, and particularly arteries. In turn, assessing and providing such information is one of the most difficult problems of current vascular fluid mechanics research.

The flow of blood/fluid is unsteady, the vessel walls are deformable, and also have complex elastic properties. Additionally, the vascular geometry can be extremely complex. Living tissue reacts to fluid mechanical changes in erratic ways, which, in turn, influences the flow properties. Huge variations in relevant parameters are known to exist from one patient to another patient (or even across time with physiological changes occurring in a single patient).

As a result, use of patient-specific models and parameters to a fullest possible extent is very desirable. Further, in vivo measurements (especially in the skull) are notoriously extremely difficult to effectuate, particularly with the precision and reliability desired in order to directly determine or verify the computed indicators.

To attempt to render these issues more tractable in ways applicable in routine clinical practice, the disclosed system may use one or more of the following techniques: estimation of complex blood flow and vessel wall interaction via the disclosed comprehensive mechanical and fluid dynamics model (such as coupled fluid structure interaction analysis using an elastic vessel wall model); huge variations in parameters from patient to patient may be accommodated via use of patient-specific parameters; vasculature geometry may be accurately determined from multidimensional image or volumetric data from measurements made on the patient; flow information may be determined by measurements on the actual patient, or may be estimated using automatically retrieved data corresponding to similar patients; and material properties may be determined using a database containing biomechanical properties measurements of real vessel wall tissue specimens. These and other variations are all ways of utilizing the information which is available or obtainable to leverage the benefits obtainable from the processes 700 through 1000 of FIGS. 7 through 10, respectively, to derive increased accuracy and robustness of patient needs, via appropriately exercising the FSI solver 110 of FIG. 1, 210 of FIG. 2 and/or 310 of FIG. 3.

The processes 700, 800, 900 and 1000 of FIGS. 7 through 10, respectively, thus provide improved, automated modeling of vascular pathologies, even in the context of ongoing medical procedures, facilitates care and intervention planning, and allows comparisons to be made to prior assessments, in order to track progress and to determine if or when further intervention may be appropriate. An example of a computer useful in implementing this type of process is described below with reference to §V.

### §V. Hardware and Operating Environment

FIG. 11 illustrates an example of a general computation resource 1100 useful in implementation of one or more of the processes 700 through 1000 of FIGS. 7 though 10, respectively, in relation to the system 100, 200, 300 shown and described above with reference to FIGS. 1 through 3, respectively, in accordance with an embodiment of the disclosed subject matter. The general computer environment 1100 includes a computation resource 1102 capable of implementing the processes described herein. It will be appreciated that other devices may alternatively used that include more components, or fewer components, than those illustrated in FIG. 11.

The illustrated operating environment 1100 is only one example of a suitable operating environment, and the example described with reference to FIG. 11 is not intended to suggest any limitation as to the scope of use or functionality of the embodiments of this disclosure. Other well-known computing systems, environments, and/or configurations may be suitable for implementation and/or application of the subject matter disclosed herein.

The computation resource 1102 includes one or more processors or processing units 1104, a system memory 1106, and a bus 1108 that couples various system components including the system memory 1106 to processor(s) 1104 and other elements in the environment 1100. The bus 1108 represents one or more of any of several types of bus structures, including a memory bus or memory controller, a peripheral bus, an accelerated graphics port and a processor or local bus using any of a variety of bus architectures, and may be compatible with SCSI (small computer system interconnect), or other conventional bus architectures and protocols.

The system memory 1106 includes nonvolatile read-only memory (ROM) 1110 and random access memory (RAM) 1112, which may or may not include volatile memory elements. A basic input/output system (BIOS) 1114, containing the elementary routines that help to transfer information between elements within computation resource 1102 and with external items, typically invoked into operating memory during start-up, is stored in ROM 1110.

The computation resource 1102 further may include a non-volatile read/write memory 1116, represented in FIG. 11 as a hard disk drive, coupled to bus 1108 via a data media interface 1117 (e.g., a SCSI, ATA, or other type of interface); a magnetic disk drive (not shown) for reading from, and/or writing to, a removable magnetic disk 1120 and an optical disk drive (not shown) for reading from, and/or writing to, a removable optical disk 1126 such as a CD, DVD, or other optical media.

The non-volatile read/write memory 1116 and associated computer-readable media provide nonvolatile storage of computer-readable instructions, data structures, program modules and other data for the computation resource 1102. For example, data recorded as described above in §IV with reference to FIGS. 8 through 10, e.g., such as noted in blocks 755, 830, 935 or 1035, may be written to the non-volatile read/write memory 1116, removable magnetic disk 1120 or removable optical disk 1126. Similarly, data which are being recalled or imported as noted in blocks 910 or 1010 or is being extracted from a database, as described above in §I with references to FIGS. 1 to 3, may be read from the non-volatile read/write memory 1116, removable magnetic disk 1120 or removable optical disk 1126.

Although the exemplary environment 1100 is described herein as employing a non-volatile read/write memory 1116, a removable magnetic disk 1120 and a removable optical disk 1126, it will be appreciated by those skilled in the art that other types of computer-readable media which can store data that is accessible by a computer, such as magnetic cassettes, FLASH memory cards, random access memories (RAMs), read only memories (ROM), and the like, may also be used in the exemplary operating environment.

A number of program modules may be stored via the non-volatile read/write memory 1116, magnetic disk 1120, optical disk 1126, ROM 1110, or RAM 1112, including an operating system 1130, one or more application programs 1132, other program modules 1134 and program data 1136. Examples of computer operating systems conventionally employed for some types of three-dimensional and/or two-dimensional medical image data include the NUCLEUS® operating system, the LINUX® operating system, and others, for example, providing capability for supporting application programs 1132 using, for example, code modules written in the C++® computer programming language.

A user may enter commands and information into computation resource 1102 through input devices such as input media 1138 (e.g., keyboard/keypad, tactile input or pointing device, mouse, foot-operated switching apparatus, joystick, touchscreen or touchpad, microphone, antenna etc.). Such input devices 1138 are coupled to the processing unit 1104 through a conventional input/output interface 1142 that is, in turn, coupled to the system bus. A monitor 1150 or other type of display device is also coupled to the system bus 1108 via an interface, such as a video adapter 1152.

The computation resource 1102 may include capability for operating in a networked environment using logical connections to one or more remote computers, such as a remote computer 1160. The remote computer 1160 may be a personal computer, a server, a router, a network PC, a peer device or other common network node, and typically includes many or all of the elements described above relative to the computation resource 1102. In a networked environment, program modules depicted relative to the computation resource 1102, or portions thereof, and/or patient records may be stored in a remote memory storage device such as may be associated with the remote computer 1160. By way of example, remote application programs 1162 reside on a memory device of the remote computer 1160. In one embodiment,

the FSI solver module 110, 210, 310 of FIGS. 1 through 3 may use the services of or reside on a remote computation server 1160 to achieve this. The remote computation server 1160 may receive problem definitions from multiple workstations through network connections, and provides rapid real-time capability for performing the resource-intensive computations needed for the FSI processing. Raw computation results are then returned to the workstation, which may be a computation resource such as the computer 1102. A computation server 1160 can be shared among multiple workstations 1102, which may be located within the same institution or on a common campus, or may be shared between multiple institutions/locations.

The logical connections represented in FIG. 11 may include interface capabilities, e.g., such as interface capabilities 152 (FIG. 1) a storage area network (SAN, not illustrated in FIG. 11), local area network (LAN) 1172 and/or a wide area network (WAN) 1174, but may also include other networks. Such networking environments are commonplace in modern computer systems, and in association with intranets and the Internet. In certain embodiments, the computation resource 1102 executes an Internet Web browser program (which may optionally be integrated into the operating system 1130), such as the “Internet Explorer®” Web browser manufactured and distributed by the Microsoft Corporation of Redmond, Wash.

When used in a LAN-coupled environment, the computation resource 1102 communicates with or through the local area network 1172 via a network interface or adapter 1176. When used in a WAN-coupled environment, the computation resource 1102 typically includes interfaces, such as a modem 1178, or other apparatus, for establishing communications with or through the WAN 1174, such as the Internet. The modem 1178, which may be internal or external, is coupled to the system bus 1108 via a serial port interface.

In a networked environment, program modules depicted relative to the computation resource 1102, or portions thereof, may be stored in remote memory apparatus. It will be appreciated that the network connections shown are exemplary, and other means of establishing a communications link between various computer systems and elements may be used.

A user of a computer may operate in a networked environment using logical connections to one or more remote computers, such as a remote computer 1160, which may be a personal computer, a server, a router, a network PC, a peer device or other common network node. Typically, a remote computer 1160 includes many or all of the elements described above relative to the computer 1100 of FIG. 11.

The computation resource 1102 typically includes at least some form of computer-readable media. Computer-readable media may be any available media that can be accessed by the computation resource 1102. By way of example, and not limitation, computer-readable media may comprise computer storage media and communication media.

Computer storage media include volatile and nonvolatile, removable and non-removable media, implemented in any method or technology for storage of information, such as computer-readable instructions, data structures, program modules or other data. The term “computer storage media” includes, but is not limited to, RAM, ROM, EEPROM, FLASH memory or other memory technology, CD, DVD, or other optical storage, magnetic cassettes, magnetic tape, magnetic disk storage or other magnetic storage devices, or any other media which can be used to store computer-intelligible information and which can be accessed by the computation resource 1102.

Communication media typically embodies computer-readable instructions, data structures, program modules or other data, represented via, and determinable from, a modulated data signal, such as a carrier wave or other transport mechanism, and includes any information delivery media. The term “modulated data signal” means a signal that has one or more of its characteristics set or changed in such a manner as to encode information in the signal in a fashion amenable to computer interpretation.

By way of example, and not limitation, communication media include wired media, such as wired network or direct-wired connections, and wireless media, such as acoustic, RF, infrared and other wireless media. The scope of the term computer-readable media includes combinations of any of the above.

As such, the computer 1102 may function as one or more of the elements shown in FIGS. 1 through 3, for example, via implementation of the processes 700, 800, 900 and/or 1000 of FIGS. 7 through 10, respectively, as one or more computer program modules. A conclusion is presented below in §VI.

### §VI. Conclusion

The disclosed examples combine a number of useful features and present advantages in modern hospital settings. These examples address, among other things, a key problem with segmenting and quantifying lesions, and particularly liver lesions, due to a lack of repeatability. The inconsistent repeatability results from a number of causes, including various inconsistencies in the contrast uptakes of the lesions due to variations in timing between contrast agent injection and/or variations in timing of the phases, and the imaging. The combination of multiple contrast-agent enhanced datasets taught by the present disclosure provides additional enhancement of the anatomy to create a more robust contrast between the lesion and the surrounding parenchyma. In turn, this tends to improve consistent segmentation and quantification that can be relied on for growth/change analysis, surgical planning, radiotherapy planning and other purposes.

Additionally, compatibility with existing tools and modes for image data representation, and conventional image data storage and exchange standards facilitate interoperability with existing modules developed for those purposes, as well as promoting compatibility with newer approaches, such as integrated surgical navigation. The disclosed capabilities also benefit from compatibility with existing systems, and thus coordinate with other operator training, reducing probability of error, such as may occur in time-critical scenarios.

Although specific embodiments have been illustrated and described herein, it will be appreciated by those of ordinary skill in the art that any arrangement which is calculated to achieve the same purpose may be substituted for the specific embodiments shown. This disclosure is intended to cover any adaptations or variations. For example, although described in procedural terms, one of ordinary skill in the art will appreciate that implementations can be made in a procedural design environment or any other design environment that provides the required relationships.

In particular, one of skill in the art will readily appreciate that the names or labels of the processes and apparatus are not intended to limit embodiments. Furthermore, additional processes and apparatus can be added to the components, functions can be rearranged among the components, and new components to correspond to future enhancements and physical devices used in embodiments can be introduced without departing from the scope of embodiments. One of skill in the art will readily recognize that embodiments are applicable to future communication devices, different file systems, and new data types. The terminology used in this disclosure is meant to include all object-oriented, database and communication environments and alternate technologies which provide the same functionality as described herein.

What is claimed is:

1. A system for characterizing aspects of vascular scenarios, comprising:
an input module;
a database for storing characteristics of various types and conditions of vascular segments, a vascular region of interest and associated environments, properties of tools associated with treatment of vascular abnormalities, and patient-related indicia, or information identifying such;
access to a FSI solver, the FSI solver for accepting an ensemble including at least some of the characteristics, conditions, a description of the vascular region of interest and associated environments, the properties of tools associated with treatment of vascular abnormalities, and the patient-related indicia, or information identifying such from the input module and the database, and using the accepted ensemble to model the region of interest and provide results from modeling the region of interest;
interfaces for transmitting information from the input module and the database to the FSI solver and for receiving the results from the FSI solver; and
a collection of analysis modules, coupled to the interface for receiving results, the collection for comparing various treatment options, allowing before-and-after comparisons of aspects of the region of interest and providing quantitative assessments of parameters of interest describing the region of interest from the results.

2. The system of claim 1, wherein the access to the FSI solver is via a bus, which fulfills, at least in part, the functions of the interfaces for transmitting and for receiving, and wherein the FSI solver is a part of the system.

3. The system of claim 1, wherein the access to the FSI solver is via a network, which fulfills, at least in part, the functions of the interfaces for transmitting and for receiving, and wherein the FSI solver is remote from other portions of the system.

4. The system of claim 1, wherein the FSI solver is operable to iteratively perform a computational fluid dynamic analysis of pulsatile fluid flow, and, with results from the fluid analysis, employ a finite-element mechanical analysis of vessel properties including deformation due to the pulsatile loading by the fluid, and then, using the results from the finite-element mechanical analysis, re-engage the computational fluid dynamic analysis of pulsatile fluid flow, followed by further finite element mechanical analysis of vessel properties, until a predetermined convergence criterion is achieved, and then to provide raw simulation data from the iteratively-performed analyses to other analysis modules for further, application-specific processing.

5. The system of claim 1, wherein results from the FSI solver are employed in a pre-operative, characterization phase, to compare benefits and drawbacks of various treatment protocols and tools and aid in selection of an appropriate one or ones of treatment modalities for implementation or further evaluation.

6. The system of claim 1, wherein results from the FSI solver are employed in a intra-operative mode, to compare present status to a planned-for result, and to determine from that comparison what actions, if any, are suggested.

7. The system of claim 1, wherein results from the FSI solver are employed in a post-operative mode, to facilitate comparison of a present risk profile to a planned result and associated risk profile, and to determine from that comparison what actions, if any, are suggested.

8. A process for characterizing aspects of vascular scenarios, comprising acts of:
accepting patient indicia via an input module;
accessing relevant data records from a database using the indicia, and augmenting those data records, where needed, with stored data from a bank of representative data also stored in the database, to provide information including a description of the vascular scenario and defining a region of interest;
sending the information to a FSI solver;
receiving, responsive to sending, raw simulation results from the FSI solver; and
modifying the raw simulation results using selected items from a collection of analysis modules, the selected items from the collection for comparing various treatment options, allowing before-and-after comparisons of aspects of the region of interest and providing quantitative assessments of parameters of interest describing the region of interest from the results.

9. The process of claim 8, wherein the act of sending includes invoking the FSI solver to accept an ensemble including at least some of:
characteristics and conditions associated with the indicia,
a description of the vascular scenario including a region of interest and associated environments,
properties of tools associated with treatment of vascular abnormalities, and
the patient-related indicia, or information identifying such; and
using the accepted ensemble to model the region of interest and provide results from modeling the region of interest.

10. The process of claim 8, wherein the act of accepting patient indicia comprises accepting indicia identifying prior assessment and simulation results for comparison to present simulation results derived from a present measurement as part of a post-treatment evaluation process.

11. The process of claim 8, further comprising importing present examination data on a continuing real-time basis as part of an intra-operative process, and comparing simulation results derived from the present examination data via the FSI solver to a planned treatment profile in order to determine what actions, if any, are warranted in order to promote achievement of the planned treatment profile.

12. The process of claim 8, wherein, following definition of a region of interest, present measured data are collected and are employed together with the information including a description of the vascular scenario by the FSI solver to provide a present simulation of the region of interest.

13. The process of claim 8, further comprising, prior to sending, optionally invoking a simplified model in order to obtain a rough estimation indicative of whether or not the values to be sent to the FSI solver via sending appear to be appropriate or appear to require adjustment prior to sending.

14. The process of claim 8, wherein the FSI solver, after sending and prior to receiving, determines, via a predetermined convergence criterion, when to terminate iteration of alternative modeling of flow simulation using computational fluid dynamics, and coupling result from flow simulation to a mechanical analysis to determine impact of the flow simulation on vasculature properties, and then employing results from the mechanical analysis to refine the modeling of flow simulation.

15. A computation engine and a memory coupled to a data collection module; and
computer-readable code embodied on a computer-readable medium and configured so that when the computer-readable code is executed by one or more processors associated with the computation engine, the computer-readable code causes the one or more processors to:
accept input indicia from the data collection module, the input indicia identifying a particular patient and allowing access to stored records relating to prior measurements and simulations, if any, relative to that patient;
determine estimates for quantities not represented in a present measurement from a database storing characteristics of various types and conditions of vascular segments associated with a defined vascular region of interest and associated environments, to determine appropriate properties of tools associated with treatment of vascular abnormalities, in conformance with patient-related indicia, or information identifying such;
access a FSI solver, the FSI solver for accepting an ensemble including at least some of the characteristics, conditions, a description of the vascular region of interest and associated environments, the properties of tools associated with treatment of vascular abnormalities, and the patient-related indicia, or information identifying such from the input module and the database, and using the accepted ensemble to model the region of interest and provide results from modeling the region of interest;
exchange information between the input module and the database and the FSI solver, including providing results from the FSI solver to a collection of analysis modules; and
using the collection of analysis modules, and the results from the FSI solver to:
compare benefits and potential drawbacks of various treatment options; or
allow before-and-after comparisons of aspects of the region of interest; or
provide quantitative assessments of parameters of interest describing the region of interest from the results.

16. The apparatus of claim 15, wherein the computer readable code is further configured so that, when executed by the one or more processors, the computer readable code configured to cause the one or more processors to compare includes causing the one or more processors to compare benefits and potential drawbacks of various treatment options as part of a pre-treatment evaluation of one or more potential treatment plans.

17. The apparatus of claim 15, wherein the computer readable code is further configured so that, when executed by the one or more processors, the computer readable code configured to cause the one or more processors to compare includes causing the one or more processors to compare benefits and potential drawbacks of various treatment options as part of a intra-treatment process for evaluation of differences between present and planned treatment profiles, and, when warranted, determine actions, if any, appropriate for correction of apparent deviations from a planned treatment profile.

18. The apparatus of claim 15, wherein the computer readable code is further configured so that, when executed by the one or more processors, the computer readable code configured to cause the one or more processors to compare includes causing the one or more processors to compare benefits and potential drawbacks of various treatment options as part of a pre-operative risk assessment and treatment planning process.

19. The apparatus of claim 15, wherein the computer readable code is further configured so that, when executed by the one or more processors, the computer readable code is configured to cause the one or more processors to evaluate convergence, using a predetermined convergence criterion, of a process involving successive iteration of a computational fluid dynamics computation of fluid flow simulation, with an output from the fluid flow simulation being coupled to a mechanical analysis of vasculature to determine variations in the vasculature as a result of the fluid flow, and with an output of the mechanical analysis of vasculature being coupled to an input to the computational fluid dynamics computation of fluid flow simulation, and to cease iteration when the convergence criteria is achieved.

20. The apparatus of claim 15, wherein the computer readable code is further configured so that, when executed by the one or more processors, the computer readable code is configured to cause the one or more processors to evaluate convergence of concatenated simulations in the FSI solver to an acceptable degree and to then supply results from the concatenated simulations to the collection of analysis modules for further processing.
